(19) United States
(12) Patent Application Publication
(10) Pub. No.: US 2010/0099896 A1
(43) Pub. Date: Apr. 22, 2010

(54) METHOD OF PREPARING DOCETAXEL AND INTERMEDIATES USED THEREIN

(75) Inventors: Nam Du Kim, Hwaseong-si (KR); Wooseob Shin, Suwon-si (KR); Jaehyuk Jung, Goyang-si (KR); Dong Jun KIm, Osan-si (JP); Gi Jeong Kim, Suwon-si (KR); Young Ho Moon, Suwon-si (KR); Young-Kil Chang, Seoul (KR); Gwan Sun Lee, Seoul (KR)

Correspondence Address: SUGHRUE MION, PLLC, 2100 PENNSYLVANIA AVENUE, N.W., SUITE 800, WASHINGTON, DC, 20037, US

(73) Assignee: HANMI PHARM. CO. LTD, Kyungki-do (KR)

(21) Appl. No.: 12/517,623

(22) PCT Filed: Dec. 3, 2007
(86) PCT No.: PCT/KR07/06190

§ 371 Date: Jun. 4, 2009

(30) Foreign Application Priority Data
Dec. 14, 2006 (KR) 10-2006-0127757

(51) Int. Cl. C07D 305/14

(52) U.S. Cl. 549/510

(57) ABSTRACT

The present invention relates to a novel method for preparing docetaxel having anti-tumor and anti-leukemia activity, and intermediates useful for preparing docetaxel.

### FIELD OF THE INVENTION

The present invention relates to a novel method of preparing docetaxel, and intermediates used therein.

### BACKGROUND OF THE INVENTION

Docetaxel of formula (I), a terpene taxane derivative, is a potent anti-tumor chemotherapeutic agent having a broad spectrum of anti-tumor and anti-leukemia activity, and has been approved as commercially marketable therapeutic agents against ovarian cancer and breast cancer.

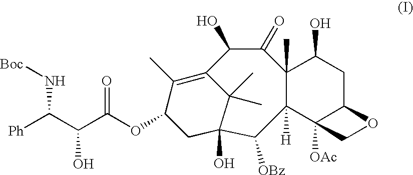

wherein,

Ph is phenyl;

Ac is acetyl;

Bz is benzoyl; and

Boc is t-butoxycarbonyl.

Hereinafter, Ph, Ac, Bz and Boc have the same meanings as defined above.

Semi-synthetic and synthetic methods for preparing docetaxel have been widely studied.

The general method of preparing docetaxel from 10-deacetylbaccatin III of formula (X), comprises 4 steps of (a) introducing a protecting group selectively into the 7- and 10-hydroxy groups of deacetylbaccatin III of formula (II); (b) subjecting the compound obtained in step (a) to a condensation reaction with an oxazolidine derivative or a salt thereof in the presence of a condensation agent; (c) opening the oxazolidine ring of the resulting product; and (d) removing the 7- and 10-hydroxy protecting groups from the compound obtained in (c).

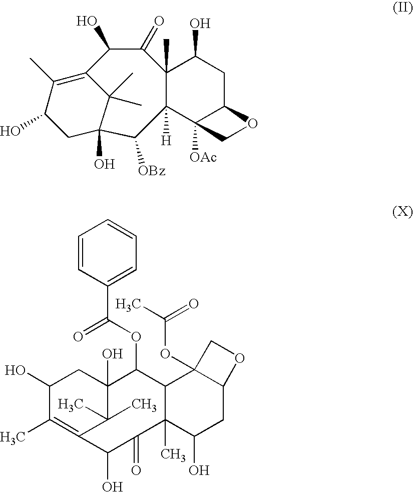

However, many known synthetic methods reported heretofore are not satisfactory in terms of their performances in step (a) of selectively introducing the protecting groups. Namely, step (a) shown in Reaction Scheme (A) does not proceed with sufficient selectivity, and it is difficult to purify the 10-deacetylbaccatin III product of formula (IVa), which when used in the preparation of docetaxel, the yield of the final product is very low and the purifying process becomes complicated.

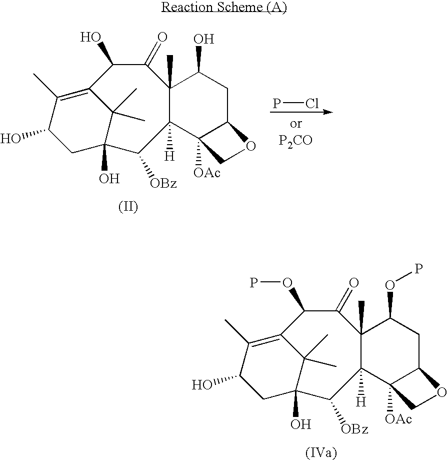

Wherein,

P is a hydroxy protecting group.

For example, as shown in Reaction Scheme (B), European Patent No. 0253738 or [Gueritte, et al., Tetrahedron, 42, 4451 (1986)] disclose a method of preparing 10-deacetylbarcatin III of formula (IVb) having protected 7- and 10-hydroxyl groups by reacting 10-deacetylbarcatin III of formula (II) with 3 equivalents of 2,2,2-trichloroethylchloroformate in pyridine at 80°, but the yield of the desired 10-deacetylbarcatin III of formula (IVb) is 85˜87%, while the 10-deacetylbarcatin III of formula (a) having protected 7-, 10- and 13-hydroxyl groups is produced in amount of 8˜12% as a byproduct. In this case, the product must be purified by column chromatography, before its use in the preparation of docetaxel.

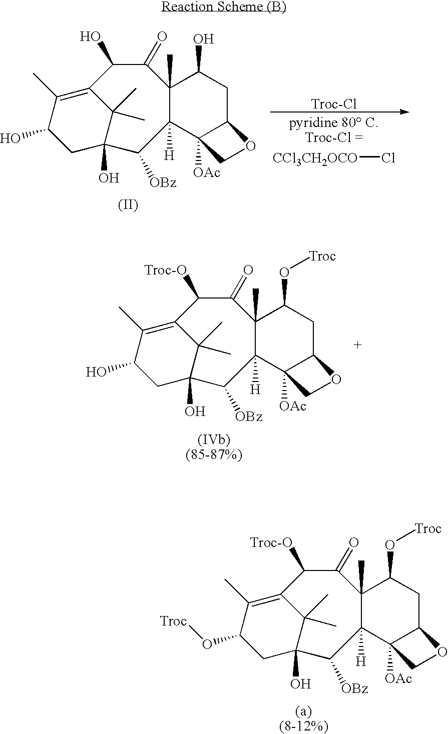

Further, as disclosed in International Patent Publication WO 04/33442 (Reaction Scheme C), the yield of 10-deacetylbaccatin III of formula (IVc) having protected 7- and 10-hydroxy groups is 83˜86%, while 10-deacetylbaccatin III of formula (b) having protected 7-, 10- and 13-hydroxy groups and 10-deacetylbaccatin III derivatives of formula (c) and (d) having protected 7- or 10-hydroxy group are co-produced as byproducts in yields of 8˜14% and 0˜3%, respectively. Therefore, this method also has a problem similar to that of European Patent No. 0253738.

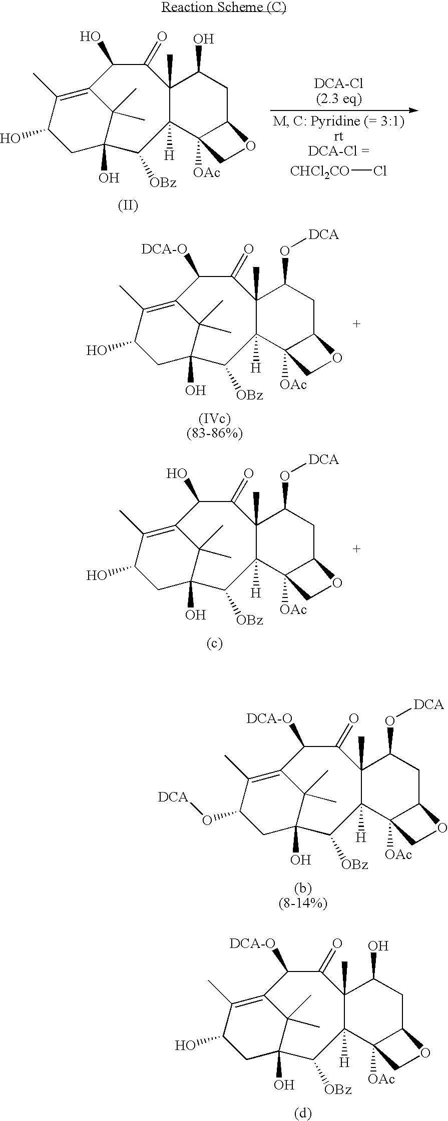

Further, as disclosed in U.S. Pat. No. 6,500,966 (Reaction Scheme D), the yield of 10-deacetylbaccatin III of formula (IVd) having protected 7- and 10-hydroxy groups prepared by using trichloroacetyl protecting group is 37˜45%, while 10-deacetylbaccatin III of formula (e) having protected 7-, 10- and 13-hydroxy groups and an unknown white solid are produced as byproducts. Therefore, this method also has a difficulty similar to those described above.

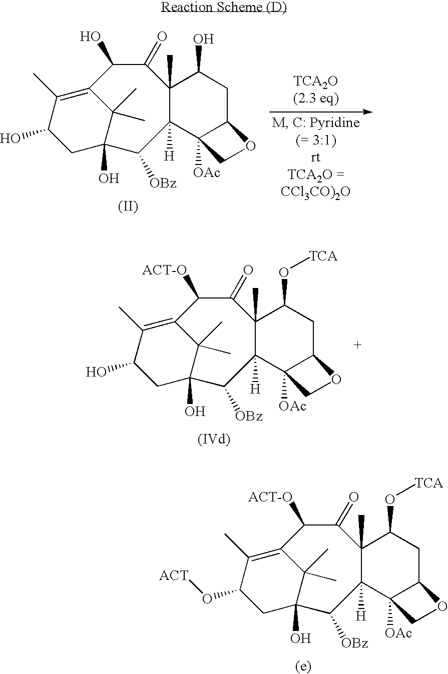

Therefore, the present inventors have attempted to prepare 10-deacetylbaccatin III having protected 7- and 10-hydroxyl groups with a high selectivity, which can be easily purified by recrystallization, and also to prepare an oxazolidine derivative which can be effectively used in the coupling reaction. As a result, the present inventors have found a novel, improved method of preparing docetaxel in a high yield.

### SUMMARY OF THE INVENTION

Accordingly, it is an object of the present invention to provide a novel method of preparing docetaxel, and intermediates used therein.

In accordance with one aspect of the present invention, there is provided a method of preparing docetaxel of formula (I), which comprises the steps of:

(i) bringing 10-deacetylbaccatin III of formula (II) to react with benzoyl halide of formula (III) in the presence of a base to obtain a compound of formula (IV) having protected 7- and 10-hydroxy groups;

(ii) subjecting the compound of formula (IV) to a coupling reaction with an oxazolidine derivative of formula (V) or a salt thereof in the presence of a condensation agent to obtain a taxane of formula (VI) having an oxazolidine side chain;

(iii) subjecting the side chain of the compound of formula (VI) to a ring opening reaction in an organic solvent in the presence of an acid to obtain the docetaxel of formula (VII) having protected 7- and 10-hydroxy groups; and

(iv) removing the protecting groups at the positions 7 and 10 of the compound of formula (VII) using a base in a solvent:

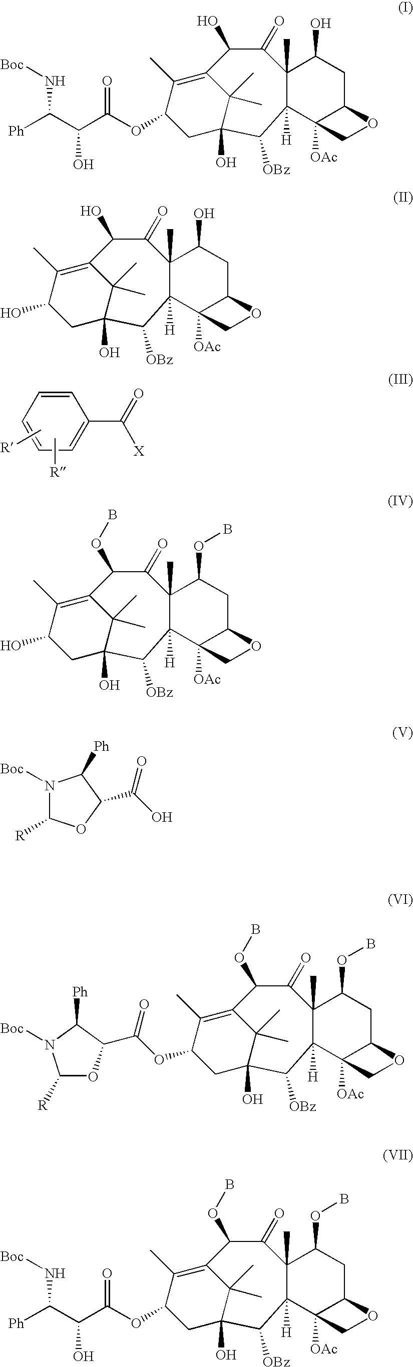

wherein,

Ph is phenyl;

Ac is acetyl;

Bz is benzoyl;

Boc is t-butoxycarbonyl;

R is 4-methoxyphenyl, isopropyl or t-butyl;

B is

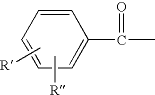

R′ and R″ are each independently hydrogen or nitro; and

X is halogen.

In accordance with another aspect of the present invention, there is provided a compound of formula (IV) which can be used as an intermediate in preparing the docetaxel of formula (I):

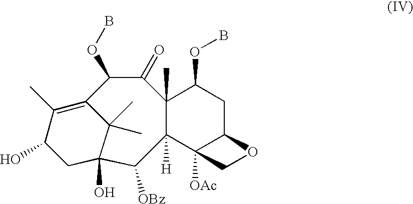

wherein,

Ac and Bz have same meanings as defined previously; and

B is 4-nitrobenzoyl, 3,5-dinitrobenzoyl, or 2,4-dinitrobenzoyl.

In accordance with a further aspect of the present invention, there is provided a compound of formula (Va) which can be used as an intermediate in preparing the docetaxel of formula (I):

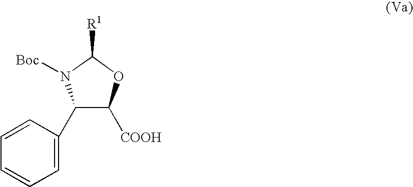

Wherein,

Boc is t-butoxycarbonyl; and

R¹ is isopropyl or t-butyl.

In accordance with a still further aspect of the present invention, there is provided a method of preparing the compound of formula (Va), which comprises the steps of:

(i) bringing (2R,3S)-3-phenylisoserine methyl ester formic acid addition salt of formula (VIII) dissolved in a solvent to react with isobutylaldehyde or trimethylacetaldehyde, and adding di-t-butyl-dicarbonate thereto to obtain an oxazolidine ring compound of formula (IX); and

(ii) subjecting the compound of formula (IX) to hydrolysis in the presence of a base:

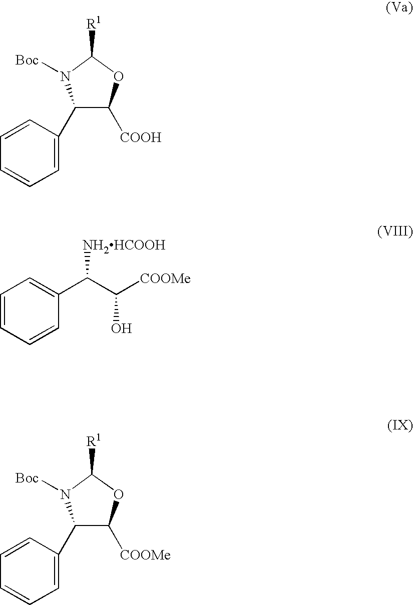

Wherein,

Boc is t-butoxycarbonyl; and

R¹ is isopropyl or t-butyl.

### DETAILED DESCRIPTION OF THE INVENTION

The method of preparing docetaxel according to the present invention is characterized by the use of both the compound of formula (IV) whose 7- and 10-hydroxy groups are selectively protected with benzoyl groups having one or more optional nitro substituents and the compound of formula (V) in the coupling reaction with the compound of formula (IV).

Docetaxel of formula (I) of the present invention may be prepared by the procedure shown in Reaction Scheme (E).

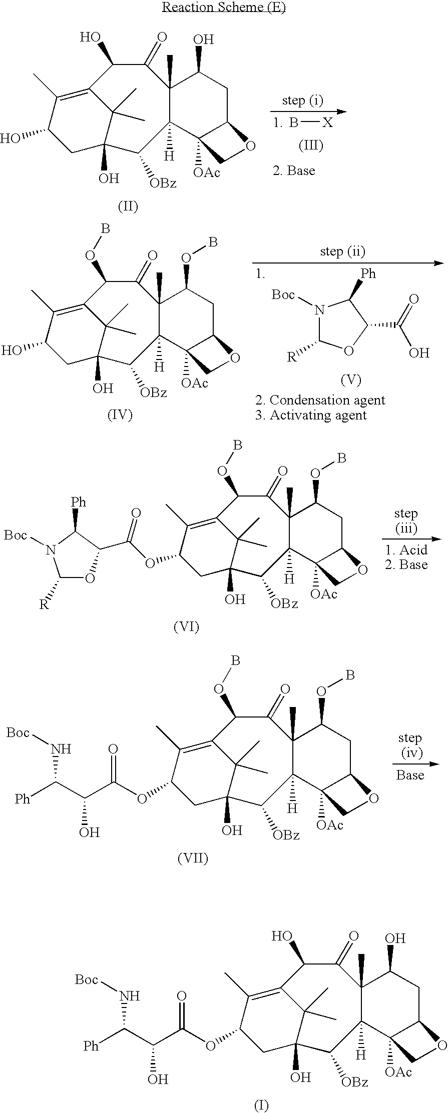

wherein,

B, X and R have the same meanings as defined previously.

In step (i), 10-deacetylbaccatin III of formula (II) is allowed to react with a benzoyl halide (B-X) of formula (III) in the presence of a base to obtain a novel compound, a compound of formula (IV) having selectively protected 7- and 10-hydroxyl groups, which is a key intermediate used in the present invention.

This reaction may be carried out at a temperature ranging from 20° to 60°, preferably. The benzoyl halide used in this reaction may be 4-nitrobenzoyl chloride, 3,5-dinitrobenzoyl chloride, or 1,4-dinitrobenzoyl chloridetoluene, preferably 3,5-dinitrobenzoyl chloride. The product can be easily purified by recrystallization from a solvent, e.g., methanol, especially when 3,5-dinitrobenzoyl chloride is used, to obtain pure deacetylbaccatin III of formula (IV) in a high yield. In this reaction, the benzoyl halide may be used in an amount of 2 to 5 equivalents based on the compound of formula (II). The base used in this reaction may be an amine such as pyridine and triethylamine, and the solvent that can be used in this reaction is chloroform, dichloromethane or ethyl acetate.

In step (ii), the compound of formula (IV) obtained in step (i) is subjected to a coupling reaction with the oxazolidine derivative of formula (V) or a salt thereof in a solvent in the presence of a condensation agent to obtain a taxane derivative of formula (VI) having an oxazolidine side chain.

This reaction may be carried out at a temperature ranging from 0° to 80°, and the compound of formula (V) may be used in an amount of 1.5 to 5 equivalents based on the compound of formula (IV). The solvent used in this reaction may be ethyl acetate, methyl acetate, chloroform, or dichloromethane, and the condensation agent used in this reaction may be dicyclohexylcarbodiimide in an amount of 1 to 5 equivalents based on the compound of formula (IV).

Further, an activating agent such as amines (e.g., 4-dimethylaminopyridine and pyridine) may be added to the reaction mixture in a less than stoichiometric amount based on the compound of formula (IV).

The taxane derivative of formula (VI) thus obtained may be recrystallized from a methanol-hexane mixture or acetonitrile-water mixture to obtain a purified form of the compound of formula (VI).

In step (iii), the compound of formula (VI) having an oxazolidine side chain obtained in step (ii) is subjected to a ring opening reaction in an organic solvent in the presence of an acid to obtain the docetaxel of formula (VII) having protected 7- and 10-hydroxy groups. The acid used in the ring opening reaction may be hydrochloric acid, sulfuric acid, formic acid or p-toluenesulfonic acid in an amount of 1 to 100 equivalents based on the compound of formula (VI). The organic solvent used in this reaction may be chloroform, ethyl acetate, methyl acetate, dichloromethane, tetrahydrofuran, and a mixture thereof.

When R is 4-methoxyphenyl, the compound of formula (VII) can be obtained without lossing the t-butoxycarbonyl group. However, when R is isopropyl or t-butyl, the t-butoxy group may be removed. In this case, the compound of formula (II) can be obtained by neutralizing the reacting solution with a suitable base, adding water thereto, and adding di-t-butyl-dicarbonate to the resulting solution. The compound of formula (II) thus obtained can be easily purified by recrystallization from a mixture of diethyl ether-hexane or a mixture of acetonitrile-water.

In step (iv), the protecting group B of the compound of formula (VII) is removed in a solvent in the presence of a base to obtain docetaxel of formula (I). In this reaction, the base may be morpholine, diethyl amine, ammonia, methylamine, or t-butyl amine which is used in an amount of 1 to 40 equivalents based on the docetaxel of formula (VII) and the solvent is preferably a C₁₋₃ alcohol, most preferably methanol.

Further, the oxazolidine derivative of formula (V) used in step (ii) of the present invention may be prepared as follows.

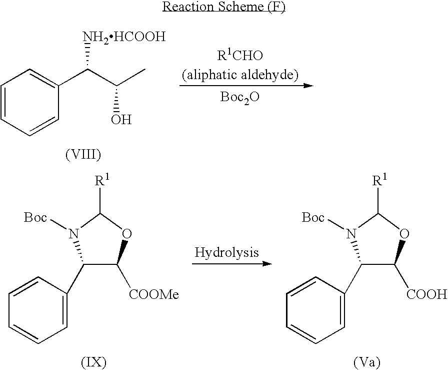

wherein,

R¹ has the same meaning as defined previously.

In this reaction, an aliphatic aldehyde (R¹CHO) such as isobutylaldehyde or trimethylacetaldehyde is added together with di-t-butyl-dicarbonate to (2R,3S)-3-phenylisoserine methyl ester formic acid addition salt of formula (VIII) dissolved in a solvent such as chloroform, ethyl acetate, methyl acetate, dichloromethane and tetrahydrofuran to obtain an oxazolidine ring compound of formula (IX) having a N-t-butyl carbonyl group. Then, the resulting compound may be subjected to hydrolysis with a suitable base to obtain the oxazolidine derivative of formula (Va), and the base used in this reaction may be lithium hydroxide, sodium hydroxide, or potassium hydroxide.

In addition, when R of the compound of formula (V) is 4-methoxyphenyl group, the compound may be prepared in accordance with Korean Patent Publication NO. 1995-0703547.

In accordance with the method of the present invention, high-purity docetaxel can be prepared in a high yield, which has not been achieved in the prior art.

The following Examples are intended to further illustrate the present invention without limiting its scope.

### Example 1

### Preparation of 7,10-(di-3′,5′-dinitrobenzoyl)-10-deacetylbaccatin III (Compound of Formula (IV))

11.0 g of 10-deacetylbaccatin III was dissolved in a mixture of 28 m [symbol] of pyridine and 55 m [symbol] of chloroform, and 11.0 g of 3,5-dinitrobenzoylchloride was slowly added dropwise thereto while keeping the reaction temperature at 38 to 42°. Then, pyridine was removed therefrom under a reduced pressure to obtain 7,10-(di-3′,5′-dinitrobenzoyl)-10-deacetylbaccatin III in a yield of 97.1% together with 0.3% of 7- or 10-(3′,5′-dinitrobenzoyl)-10-deacetylbaccatin III and 0.9% of 7,10,13-(tri-3′,5′-dinitrobenzoyl)-10-deacetylbaccatin III as byproducts. A mixture of 110 m [symbol] of methanol and 50 m [symbol] of 1 N HCl was added dropwise to the residue, vigorously stirred for 1 hour and filtered. The resulting solid was added to 220 m [symbol] of methanol, stirred at room temperature for 1 hour and filtered. The solid was then dried to obtain the title compound (16.9 g; yield: 93%; purity: 98.5%).

b.p.: 234°;

a_{D}²³=−77.8° (c=1, CHCl₃);

IR (KBr, cm⁻¹) 3458, 3103, 2949, 1739, 1717, 1628, 1600, 1550, 1453, 1343, 1273, 1167, 1109, 1071, 973, 918, 835, 716;

¹H-NMR (CDCl₃, 300MHz) δ 9.27 (m, 1H), 9.20 (m, 1H), 9.04 (m, 2H), 8.77 (m, 2H), 8.14 (d, J=7.5 Hz, 2H), 7.50-7.68 (m, 3H), 6.65 (s, 1H), 5.94 (dd, J=7.0 Hz, J=10.0 Hz, 1H), 5.74 (d, J=7.0 Hz, 1H), 5.07 (d, J=9.0 Hz, 1H), 4.94-4.98 (m, 1H), 4.41 (d, J=8.4 Hz, 1H), 4.23 (d, J=8.4 Hz, 1H), 4.13 (d, J=6.7 Hz, 1H), 2.84-2.95 (m, 1H), 2.35-2.42 (m, 2H), 2.37 (s, 3H), 2.21 (s, 3H), 1.99-2.07 (s, 1H), 2.00 (s, 3H), 1.27 (s, 3H), 1.23 (s, 3H);

Elemental analysis: C₄₃H₄₀N₄O₂₀;

Theoretical value: C, 55.37; H, 4.32; N, 6.01; and

Experimental value: C, 55.42H, 4.29 N, 6.13.

### Example 2

### Preparation of (2R,4S,5R)-3-t-butoxycarbonyl-2-isopropyl-4-phenyl-1,3-oxazolidine-5-carboxylic acid (Compound of Formula (Va))

(2-1) Preparation of (2R,4S,5R)-3-t-butoxycarbonyl-2-isopropyl-4-phenyl-1,3-oxazolidine-5-carbonyl methyl ester

5 g of (2R,3S)-3-phenyl isoserine methyl ester formate was dissolved in a mixture of 1.6 m [symbol] of pyridine and 100 m [symbol] of CHCl₃. 1.8 m [symbol] of isobutylaldehyde was added dropwise thereto and stirred for 2 hours while keeping the solution temperature at 50° C. The resulting solution was cooled to room temperature. 3 g of NaHCO₃ was added in small portions thereto and stirred for 1 hour. The solid in the reaction mixture was filtered off, and 4.4 g of di-t-butyl-dicarbonate was slowly added dropwise to the filtrate. The resulting solution was stirred at room temperature for 12 hours, and the solvent was removed under a reduced pressure to obtain an unpurified form of the title compound (7.24 g; yield: 100%).

aD²³=−24.0° (c=1, CHCl₃);

IR (KBr, cm⁻¹)3499, 3395, 3090, 3064, 3032,4 2974, 2933, 2876, 1757, 1705, 1456, 1367, 1253, 1164, 1115, 1092, 1018, 942, 849, 764, 698, 600, 465,

¹H-NMR (CDCl₃, 300 MHz) δ 7.27-7.40 (m, 5H), 5.32 (d, J=7.1 Hz, 1H), 5.20 (b, 1H), 4.73 (d, J=4.7 Hz, 1H), 3.81 (s, 3H), 2.00 (m, 1H), 1.40 (s, 9H), 1.06 (d, J=6.8 Hz 3H), 0.97 (d, J=6.8 Hz, 3H);

Elemental analysis: C₁₉H₂₇N₁O₅;

Theoretical value: C, 65.31; H, 7.79; N, 4.01; and

Experimental value: C, 65.43H, 7.71 N, 4.12.

(2-2) Preparation of (2R,4S,5R)-3-t-butoxycarbonyl-2-isopropyl-4-phenyl-1,3-oxazolidine-5-carboxylic acid

7 g of the compound obtained in (2-1) was dissolved in 35 m [symbol] of methanol. 7 m [symbol] of 3N lithium hydroxide was added dropwise thereto and stirred at room temperature for 2 hours. 20 m [symbol] of methanol was removed therefrom under a reduced pressure, and 20 m [symbol] of water was added thereto. The water-methanol solution was washed twice with 30 m [symbol] portions of hexane, and 40 m [symbol] of ethyl acetate was added thereto at 0°. The resulting mixture was neutralized by adding 7 m [symbol] of 3 N HCl while vigorously stirring. The organic layer was separated and the aqueous layer was extracted with 40 m [symbol] of ethyl acetate. The combined organic layer was dried over anhydrous sodium sulfate, filtered, and the organic solvent was removed from the filtrate under a reduced pressure to obtain the title compound (4.7 g; yield: 98.5%).

aD²³=+0.1° (solvent CHCl₃C=1);

IR (KBr, cm⁻¹) 3065, 3034, 2974, 2934, 2876, 1757, 1706, 1673, 1470, 1456, 1368, 1255, 1164, 1093, 1003, 941, 848, 762, 699, 595, 464;

¹H-NMR (CDCl₃, 300 MHz) δ 9.65 (brs, 1H), 7.14-7.54 (m, 5H), 5.35 (d, J=7.2 Hz, 1H), 5.27 (b, 1H), 4.75 (d, J=4.8 Hz, 1H), 2.00 (m, 1H), 1.42 (s, 9H), 1.07 (d, J=6.8 Hz 3H), 0.98 (d, J=6.8 Hz, 3H);

Elemental analysis: C₁₈H₂₅N₁O₅;

Theoretical value: C, 64.46; H, 7.51; N, 4.18; and

Experimental value: C, 64.43H, 7.48 N, 4.19.

### Example 3

### Preparation of (2R,4S,5R)-3-t-butoxycarbonyl-2-t-butyl-4-phenyl-1,3-oxazolidine-5-carboxylic acid (Compound of Formula (Va))

(3-1) Preparation of (2R,4S,5R)-3-t-butoxylcarbonyl-2-(4-t-butyl)-4-phenyl-1,3-oxazolidine-5-carbonyl methyl ester

The procedure of (2-1) of Example 2 was repeated except for using trimethylacetaldehyde instead of isobutylaldehyde, to obtain the title compound (7.53 g; yield: 100%).

a_{D}²³=+0.5° (c=1, CHCl₃);

IR (KBr, cm⁻¹) 3063, 2974, 2934, 1756, 1710, 1480, 1451, 1367, 1351, 1254, 1163, 948, 879, 778, 697;

¹H-NMR (CDCl₃, 300 MHz) δ 7.27-7.44 (m, 5H), 5.46 (s, 1H), 5.41 (d, J=4.2 Hz, 1H), 4.73 (d, J=4.2 Hz, 1H), 3.83 (s, 3H), 1.47 (s, 9H), 0.83 (s, 3H);

Elemental analysis: C₂₀H₂₉N₁O₅;

Theoretical value: C, 66.09; H, 8.04; N, 3.85; and

Experimental value: C, 66.13H, 7.98 N, 3.88.

(3-2) Preparation of (2R,4S,5R)-3-t-butoxycarbonyl-2-t-butyl-4-phenyl-1,3-oxazolidine-5-carboxylic acid

The procedure of (2-2) of Example 2 was repeated except for using (2R,4 S,5R)-3-t-butoxycarbonyl-2-(4-t-butyl)-4-phenyl-1,3-oxazolidine-5-carbonyl methyl ester prepared in (3-1), to obtain the title compound (2.9 g; yield: 41%).

a_{D}²³=+20.2° (c=1, CHCl₃);

IR (KBr, cm⁻¹) 31679, 3064, 2975, 2910, 1711, 1497, 1480, 1368, 1256, 1163, 1098, 1032, 948, 881, 758, 697;

¹H-NMR (CDCl₃, 300 MHz) δ 9.65 (brs, 1H), 7.54-7.14 (m, 5H), 5.35 (d, J=7.2 Hz, 1H), 5.27 (s, 1H), 4.75 (d, J=4.8 Hz, 1H), 2.00 (m, 1H), 1.42 (s, 9H), 1.07 (d, J=6.8 Hz 3H), 0.98 (d, J=6.8 Hz, 3H);

Elemental analysis: C₁₉H₂₇N₁O₅;

Theoretical value: C, 65.31; H, 7.79; N, 4.01; and

Experimental value: C, 65.33H, 7.68 N, 4.12.

### Example 4

### Preparation of Docetaxel (Compound of Formula (I))

(4-1) Preparation of (2R,4S,5R)-3-t-butoxycarbonyl-2-(4-methoxyphenyl)-4-phenyl-1,3-oxazolidine-5-carbonyl 7,10-(di-3″,5″-dinitrobenzoyl)-10-deacetylbaccatin III (compound of formula (VI))

A solution obtained by dissolving 9.3 g of 7,10-(di-3′,5′-dinitrobenzoyl)-10-deacetylbaccatin III obtained in Example 1, 10 g of (2R,4S,5R)-3-t-butoxycarbonyl-2-(4-methoxyphenyl)-4-phenyl-1,3-oxazolidine-5-carboxylic acid (prepared in accordance with International Patent Publication No. WO 1994/07878) and 61 mg of 4-(dimethylamino)pyridine in 180 m [symbol] of ethyl acetate was stirred while keeping the temperature at 40°. Then 5.2 g of dicyclohexylcarbodiimide was added thereto and stirred for 30 min, followed by the separation of the resulting dicyclohexylurea by filtration. The resulting cake was washed with 20 m [symbol] of ethyl acetate, and the combined organic layer was sequentially washed with 30 m [symbol] of 1N hydrochloric acid and 30 m [symbol] of saturated sodium bicarbonate, and dried over anhydrous MgSO₄, filtered and the organic solvent was removed from the filtrate under a reduced pressure. A mixture of 13 m [symbol] of methanol and 130 m [symbol] of hexane was added to the residue, stirred for 3 hours and filtered. 130 m [symbol] of acetonitrile and 65 m [symbol] of water was added to the resulting residue and the mixture was stirred for 3 hours and filtered to obtain the title compound (13.1 g; yield: 100%).

¹H-NMR (CDCl₃, 300 MHz) δ 9.26 (m, 1H), 9.20 (m, 1H), 9.01 (m, 2H), 8.74 (m, 2H), 8.05 (d, J=7.5 Hz, 2H), 7.66 (m, 1H), 7.53 (m, 2H), 7.50 (m, 8H), 6.93 (m, d=8.4 Hz), 6.52 (s, 1H), 6.40 (m, 1H), 6.16 (m, 1H), 5.78-5.84 (m, 1H), 5.73 (d, J=7.2 Hz), 5.45 (m, 1H), 4.96 (d, J=8.5 Hz), 4.61 (d, J=5.0 Hz, 1H), 4.32 (d, J=8.6 Hz, 1H), 4.17 (d, J=8.6 Hz, 1H), 3.95 (d, J=7.1 Hz, 1H), 3.80 (s, 3H), 2.82-2.86 (m, 1H), 2.14-2.27 (m, 2H), 1.95-2.04 (m, 2H), 1.95 (s, 3H), 1.65 (s, 3H), 1.59 (s, 3H), 1.34 (s, 3H), 1.26 (s, 3H), 1.05 (s, 9H);

Elemental analysis: C₆₅H₆₃N₅O₂₅;

Theoretical value: C, 59.40; H, 4.83; N, 5.33; and

Experimental value: C, 59.35H, 4.86 N, 5.42.

(4-2) Preparation of 13-[(2′R,3′S)-3-t-butoxycarbonylamino-3-phenyl-2-hydroxypropionyl] 7,10-(di-3″,5″-dinitrobenzoyl)-10-deacetylbaccatin III (Compound of Formula (VII))

13.1 g of (2R,4S,5R)-3-t-butoxycarbonyl-2-(4-methoxyphenyl)-4-phenyl-1,3-oxazolidine-5-carbonyl 7,10-(di-3″,5″-dinitrobenzoyl)-10-deacetylbaccatin III obtained in (4-1) was dissolved in 130 m [symbol] of ethyl acetate. 60 [symbol] of 37 (w/w) % aqueous HCl solution was added thereto and stirred at room temperature for 3 hours. The organic layer was separated and washed with 20 m [symbol] of saturated sodium bicarbonate, and dried over anhydrous MgSO₄, filtered and the organic solvent was removed from the filtrate under a reduced pressure. The resulting solid was dissolved in 120 m [symbol] of diethyl ether, and 240 m [symbol] of hexane was slowly added dropwise thereto. The mixture was stirred at room temperature for 3 hours and then filtered. The resulting solid was dissolved in 33 m [symbol] of acetonitrile, and 77 m [symbol] of water was slowly added dropwise thereto. The resulting solution was stirred at room temperature for 3 hours, and the solvent was removed by filtration to obtain the title compound (10.8 g; yield: 91%).

b.p.: 173°;

a_{D}²³=−8.9° (c=1, CHCl₃);

IR (KBr, cm⁻¹) 3543, 3432, 3101, 2978, 2900, 1736, 1628, 1548, 1494, 1455, 1368, 1345, 1269, 1163, 1095, 1070, 978, 920, 730, 718;

¹H-NMR (CDCl₃, 300 MHz) δ 9.27 (m, 1H), 9.21 (m, 1H), 9.03 (m, 2H), 8.87 (m, 2H), 8.15 (d, J=7.5 Hz, 2H), 7.65 (m, 1H), 7.54 (m, 2H), 7.40-7.43 (m, 5H), 6.63 (s, 1H), 6.27 (m, 1H), 5.88 (m, 1H), 5.80 (d, J=6.9 Hz, 1H), 5.38 (d, J=9.4 Hz, 1H), 5.28 (m, 1H), 5.03 (d, J=8.1 Hz, 1H), 4.67 (d, J=3.1 Hz, 1H), 4.41 (d, J=8.6 Hz, 1H), 4.26 (d, J=8.6 Hz, 1H), 4.07 (d, J=6.7 Hz, 1H), 3.34 (d, J=5.3 Hz, 1H), 2.87 (m, 1H), 2.46 (s, 3H), 2.42 (m, 2H), 2.01-2.05 (m, 3H), 2.01 (s, 3H), 1.87 (s, 1H), 1.59 (s, 3H), 1.39 (s, 3H), 1.36 (s, 9H), 1.32 (s, 3H);

Elemental analysis: C₅₇H₅₇N₅O₂₄;

Theoretical value: C, 57.24; H, 4.80; N, 5.86; and

Experimental value: C, 57.21H, 4.88 N, 5.90.

### (4-3) Preparation of Docetaxel

6 g of 13-[(2′R,3′S)-3-t-butoxycarbonylamino-3-phenyl-2-hydroxypropionyl] 7,10-(di-3″,5″-dinitrobenzoyl)-10-deacetylbaccatin III obtained in (4-2) was added to a mixture of 30 m [symbol] of methanol and 6 m [symbol] of morpholine, and the resulting mixture was stirred at room temperature for 3 hours. 50 m [symbol] of ethyl acetated was added dropwise thereto, and then 70 m [symbol] of 1 N HCl was slowly added dropwise thereto at 0°. The organic layer was separated and dried over anhydrous MgSO₄, filtered and the organic solvent was removed from the filtrate under a reduced pressure. The resulting residue was subjected to silica column chromatography to obtain the title compound as a white solid (3.6 g; yield; 90%).

b.p.: 195°;

a_{D}²³=−43.9° (c=0.74, ethanol);

IR (KBr, cm⁻¹) 3652, 3487, 3367, 2978, 2936, 2903, 1711, 1603, 1498, 1367, 1267, 1244, 1175, 1093, 1071, 1023, 976, 896, 709; and

¹H-NMR (CDCl₃, 300 MHz) δ 8.11 (d, J=7.2 Hz, 2H), 7.61 (m, 1H), 7.51 (m, 2H), 7.28-7.42 (m, 5H), 6.23 (m, 1H), 5.69 (d, J=7.0 Hz, 1H), 5.45 (d, J=9.6 Hz, 1H), 5.29 (m, 1H), 5.22 (s, 1H), 4.96 (m, 1H), 4.64 (m, 1H), 4.33 (d, J=8.4 Hz, 1H), 4.19-4.24 (m, 3H), 3.93 (d, J=6.9 Hz, 1H), 3.37 (d, J=5.4 Hz, 1H), 2.56 2.65 (m, 1H), 2.39 (s, 3H), 2.27-3.1 (m, 2H), 1.82-1.91 (m, 1H), 1.86 (s, 3H), 1.78 (s, 3H), 1.70 (s, 1H), 1.54 (b, 1H), 1.36 (s, 9H), 1.26 (s, 3H), 1.15 (s, 9H).

### Example 5

### Preparation of Docetaxel (Compound of Formula (I))

(5-1) Preparation of (2R,4S,5R)-3-t-butoxycarbonyl-2-isopropyl-4-phenyl-1,3-oxazolidine-5-carbonyl 7,10-(di-3″,5″-dinitrobenzoyl)-10-deacetylbaccatin III (compound of formula (VI))

The procedure of (4-1) of Example 4 was repeated except for using (2R,4S,5R)-3-t-butoxycarbonyl-2-isopropyl-4-phenyl-1,3-oxazolidine-5-carboxylic acid obtained in Example 2 instead of (2R,4S,5R)-3-t-butoxycarbonyl-2-(4-methoxyphenyl)-4-phenyl-1,3-oxazolidine-5-carboxylic acid as an oxazolidine derivative, to obtain the title compound (6.3 g; yield: 100%).

b.p.: 234°;

a_{D}²³=−52.9° (c=1, CHCl₃);

IR (KBr, cm⁻¹) 3446, 3103, 2975, 2901, 1738, 1719, 1629, 1599, 1548, 1458, 1344, 1269, 1163, 1095, 1072, 1004, 981, 920, 836, 729, 718;

¹H-NMR (CDCl₃, 300 MHz) δ 9.27 (m, 1H), 9.21 (m, 1H), 9.03 (m, 2H), 8.76 (m, 2H), 8.09 (d, J=7.2 Hz, 2H), 7.68 (m, 1H), 7.54 (m, 2H), 7.40 (m, 5H), 6.63 (s, 1H), 6.33 (m, 1H), 5.88 (m, 1H), 5.78 (d, J=7.1 Hz, 1H), 5.31 (d, J=6.9 Hz, 1H), 5.26 (d, J=5.1 Hz, 1H), 4.98 (d, J=9.4 Hz, 1H), 4.75 (d, J=5.1 Hz, 1H), 4.36 (d, J=8.5 Hz, 1H), 4.21 (d, J=8.5 Hz, 1H), 4.05 (d, J=7.2 Hz, 1H), 2.82-2.90 (m, 1H), 2.20-2.36 (m, 2H), 1.95-2.04 (m, 2H), 2.09 (s, 3H), 2.03 (s, 3H), 1.99 (s, 3H), 1.83 (s, 1H), 1.40 (s, 9H), 1.30 (s, 3H), 1.26 (s, 3H), 1.12 (d, J=6.9 Hz, 3H), 1.04 (d, J=6.9 Hz, 3H);

Elemental analysis: C₆₁H₆₃N₅O₂₄;

Theoretical value: C, 58.60H, 5.08 N, 5.60; and

Experimental value: C, 58.49H, 5.07 N, 5.68.

(5-2) Preparation of 13-[(2′R,3′ S)-3-t-butoxycarbonylamino-3-phenyl-2-hydroxypropionyl] 7,10-(di-3″,5″-dinitrobenzoyl)-10-deacetylbaccatin III (Compound of Formula (VII))

A solution obtained by dissolving 6.3 g of (2R,4S,5R)-3-t-butoxycarbonyl-2-isopropyl-4-phenyl-1,3-oxazolidine-5-carbonyl 7,10-(di-3″,5″-dinitrobenzoyl)-10-deacetylbaccatin III prepared in (5-1) in a mixture of 63 m [symbol] of formic acid, 6.3 m [symbol] of methanol and 32 m [symbol] of chloroform was stirred at room temperature for 12 hours, and formic acid was distilled under a reduced pressure to obtain a solid. 63 m [symbol] of ethyl acetate was added dropwise to the residue. The organic layer was separated and washed with 60 m [symbol] of saturated sodium bicarbonate, and 1.3 g of di-t-butyl-dicarbonate was added dropwise thereto. The mixture was stirred at room temperature for 12 hours, and distilled under a reduced pressure to remove the organic solvent. The resulting solid was dissolved in 60 m [symbol] of diethyl ether, and 120 m [symbol] of hexane was slowly added dropwise thereto. The resulting mixture was stirred at room temperature for 3 hours and filtered to obtain solid. The resulting solid was dissolved in 16 m [symbol] of acetonitrile, and 32 m [symbol] of water was slowly added dropwise thereto. The resulting solution was stirred at room temperature for 3 hours, and the solvent was removed by filtration to obtain the title compound (4.2 g; yield: 70%).

### (5-3) Preparation of Docetaxel (Compound of Formula (I))

The procedure of (4-3) of Example 4 was repeated except for using the compound prepared in (5-2) as a starting material, to obtain the title compound (2.4 g; yield: 85%).

### Example 6

### Preparation of Docetaxel (Compound of Formula (I))

(6-1) Preparation of (2R,4S,5R)-3-t-butoxycarbonyl-2-t-butyl-4-phenyl-1,3-oxazolidine-5-carbonyl 7,10-(di-3″,5″-dinitrobenzoyl)-10-deacetylbaccatin III (Compound of Formula (VI))

The procedure of (4-1) of Example 4 was repeated except for using (2R,4 S,5R)-3-t-butoxycarbonyl-2-t-butyl-4-phenyl-1,3-oxazolidine-5-carboxylic acid obtained in Example 3 instead of (2R,4S,5R)-3-t-butoxycarbonyl-2-(4-methoxyphenyl)-4-phenyl-1,3-oxazolidine-5-carboxylic acid as an oxazolidine derivative, to obtain the title compound (3.2 g; yield: 100%).

b.p.: 235°;

a_{D}²³=−6.4° (c=1, CHCl₃);

IR (KBr, cm⁻¹) 3445, 3105, 2975, 1740, 1718, 1628, 1549, 1458, 1344, 1269, 1163, 1094, 1070, 978, 729, 718;

¹H-NMR (CDCl₃, 300 MHz) δ 9.28 (m, 1H), 9.21 (m, 1H), 9.03 (m, 2H), 8.77 (m, 2H), 8.11 (d, J=7.3 Hz, 2H), 7.68 (m, 1H), 7.56 (m, 2H), 7.37-7.47 (m, 5H), 6.65 (s, 1H), 6.36 (m, 1H), 5.91 (m, 1H), 5.80 (d, J=7.0 Hz, 1H), 5.50 (d, J=5.7 Hz, 1H), 5.46 (s, 1H), 5.03 (m, 2H), 4.39 (d, J=8.6 Hz, 1H), 4.23 (d, J=8.6 Hz, 1H), 4.08 (d, J=7.0 Hz, 1H), 2.86-2.89 (m, 1H), 2.23-2.41 (m, 2H), 2.13 (s, 3H), 2.12 (s, 3H), 2.09-2.04 (m, 1H), 2.00 (s, 3H), 1.88 (s, 1H), 1.44 (s, 9H), 1.41 (s, 3H), 1.32 (s, 3H), 0.93 (s, 9H));

Elemental analysis: C₆₂H₆₅N₅O₂₄;

Theoretical value: C, 58.90; H, 5.18; N, 5.54; and

Experimental value: C, 58.86H, 5.23 N, 5.60.

(6-2) Preparation of 13-[(2′R,3′S)-3-t-butoxycarbonylamino-3-phenyl-2-hydroxypropionyl] 7,10-(di-3″,5″-dinitrobenzoyl)-10-deacetylbaccatin III (Compound of Formula (VII))

The procedure of (5-2) of Example 5 was repeated except for using the compound prepared in (6-1) as a starting material, to obtain the title compound (2.5 g; yield: 83%).

### (6-3) Preparation of Docetaxel (Compound of Formula (I))

The procedure of (4-3) of Example 4 was repeated except for using the compound obtained in (6-2) as a starting material to obtain the title compound (1.5 g; yield: 90%).

Analytic and spectrometric results obtained for the docetaxel products of Examples 5 and 6 were identical with those of the compound of Example 4.

According to the present invention, 10-deacetylbaccatin III having protected 7- and 10-hydroxy groups can be prepared in a 98% as higher purity by highly selectively protecting the 7- and 10-hydroxy groups of 10-deacetylbaccatin III by introducing 3,5-dinitrobenzoylchloride thereinto, and easily removing byproducts therefrom by recrystallization from methanol.

The above results are compared with those obtained with the conventional protecting groups mentioned in Reaction Schemes (II) to (IV) in Table 1.

TABLE 1

| | 10-deacetylbaccatin | 10-deacetylbaccatin | 10-deacetylbaccatin |
|---|---|---|---|
| | III having protected | III having protected | III having protected |
| | 7- and 10- | 7 or 10- | 7-, 10- and 13- |
| Protecting Group | dihydroxy groups (%) | monohydroxy group (%) | trihydroxy groups (%) |
| 3,5-dinitrobenzoyl | 97.1 | 0.3 | 0.9 |
| 2,2,2,-trichloroethoxy | 86.0 | 0.5 | 10.5 |
| carbonyl | | | |
| Dichloroacetyl | 84.4 | 1.8 | 10.7 |
| Trichloroacetyl | 40.0 | 0.5 | 7.7 |

As shown in Table 1, the method of preparing docetaxel of the present invention gives a markedly higher yield as compared with the conventional methods.

While the invention has been described with respect to the above specific embodiments, it should be recognized that various modifications and changes of the invention also fall within the scope of the present invention defined by the claims that follow.

1-12. (canceled)

13. A method of preparing docetaxel of formula (I), which comprises the steps of:
(i) bringing 10-deacetylbaccatin III of formula (II) to react with benzoyl halide of formula (III) in the presence of a base to obtain a compound of formula (IV) having protected 7- and 10-hydroxy groups;
(ii) subjecting the compound of formula (IV) to a coupling reaction with an oxazolidine derivative of formula (V) or a salt thereof in the presence of a condensation agent to obtain a taxane of formula (VI) having an oxazolidine side chain;
(iii) subjecting the side chain of the compound of formula (VI) to a ring opening reaction in an organic solvent in the presence of an acid to obtain the docetaxel of formula (VII) having protected 7- and 10-hydroxy groups; and
(iv) removing the protecting groups at the positions 7 and 10 of the compound of formula (VII) using a base in a solvent: 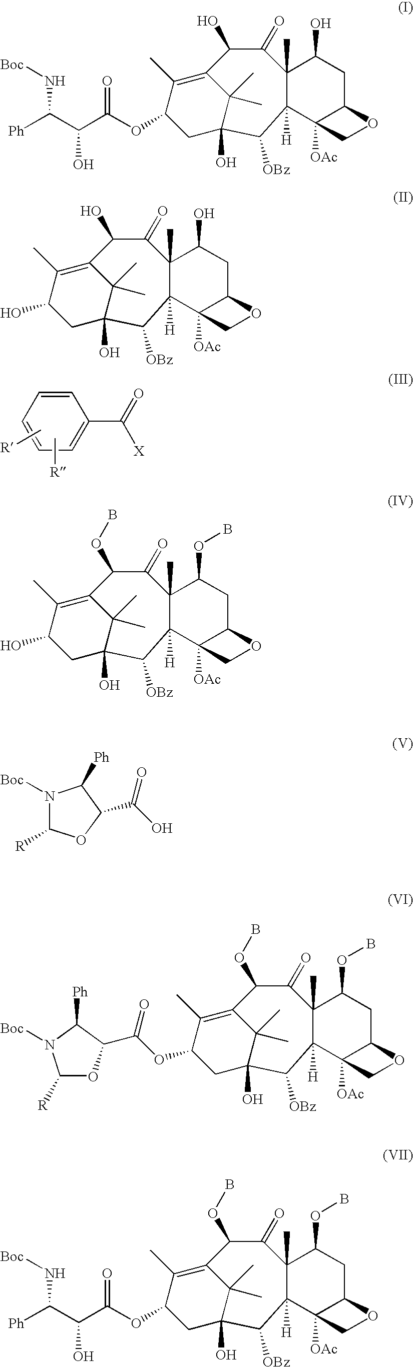
wherein,
Ph is phenyl;
Ac is acetyl;
Bz is benzoyl;
Boc is t-butoxycarbonyl;
R is 4-methoxyphenyl, isopropyl or t-butyl;
B is 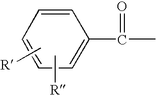
R′ and R″ are each independently hydrogen or nitro; and
X is halogen.

14. The method of claim 13, wherein the benzoyl halide of formula (III) used in step (i) is 4-nitrobenzoyl chloride, 3,5-dinitrobenzoyl chloride or 1,4-dinitrobenzoyl chloride.

15. The method of claim 13, wherein the amount of the benzoyl halide of formula (III) used in step (i) is 2 to 5 equivalents based on 10-deacetylbaccatin III.

16. The method of claim 13, wherein the base used in step (i) is pyridine or triethylamine.

17. The method of claim 13, wherein the condensation agent used in step (ii) is dicyclohexylcarbodiimide.

18. The method of claim 13, wherein 4-dimethylaminopyridine or pyridine is further added during step (ii) as an activating agent.

19. The method of claim 13, wherein the acid used in step (iii) is hydrochloric acid, sulfuric acid, formic acid, or p-toluenesulfonic acid.

20. The method of claim 19, wherein the amount of the acid used in step (iii) is 1 to 100 equivalents based on the compound of formula (VI).

21. The method of claim 13, wherein the base used in step (iv) is morpholine, diethylamine, ammonia, methylamine, or t-butyl amine.

22. A compound of formula (IV): 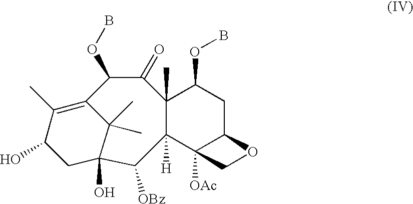
wherein,
Ac is acetyl;
Bz is benzoyl; and
B is 4-nitrobenzoyl, 3,5-dinitrobenzoyl, or 2,4-dinitrobenzoyl.
